(19) United States
(12) Patent Application Publication
(10) Pub. No.: US 2007/0140419 A1
(43) Pub. Date: Jun. 21, 2007

(54) METHOD AND APPARATUS FOR COMBINING IMAGES

(76) Inventor: Henri Souchay, Versailles (FR)

Correspondence Address: GENERAL ELECTRIC CO.;GLOBAL PATENT OPERATION, 187 Danbury Road, Suite 204, Wilton, CT, 06897-4122, US

(21) Appl. No.: 11/530,251

(22) Filed: Sep. 8, 2006

(30) Foreign Application Priority Data
Sep. 13, 2005 (FR) 05 052755

(51) Int. Cl. A61B 6/04

(52) U.S. Cl. 378/37

(57) ABSTRACT

An X-ray apparatus has a tube fitted out with an X-ray emitting focus that emits intensities of X-radiation crossing the object for a multiplicity of preliminarily determined main directions of emission, along a path. The apparatus shifts the X-ray tube along a path relative to the object. The apparatus has an X-ray detector that acquires a multiplicity of data of X-ray image data representing the multiplicity of main directions of emission. The apparatus distributes the preliminarily determined intensities of X-radiation non-uniformly on the multiplicity of main directions of emission. The apparatus also processes the multiplicity of data of X-ray image data in order to obtain both a 2D image and a 3D image of the object.

### CROSS-REFERENCE TO RELATED APPLICATIONS

This application claims the benefit of a priority under 35 USC 119(a)-(d) to French Patent Application No. 05 052755 filed Sep. 13, 2005 the entire contents of which are hereby incorporated by reference.

### BACKGROUND OF THE INVENTION

The present invention is directed to a method and apparatus for radiological imaging and in particular to an X-ray apparatus which, in an exemplary embodiment, is a mammography apparatus. The method and apparatus can be applied to but not exclusively in medical imaging and in non-destructive X-ray controls.

An embodiment of the present the invention acquires at least one radiography image projection at the same time as a series of projections for tomosynthesis processing. An embodiment of the present invention improves the ergonomic of use of an X-ray apparatus to make it both simpler and faster to use. An embodiment of present invention is also directed to distributing intensities of X-radiation non-uniformly between conventional imaging and tomosynthesis image sequences.

A conventional mammography apparatus is designed to acquire radiography images of a patient's breasts. Structurally, and by virtue of its principle, a mammography apparatus has a column that is vertical but can be oriented several times obliquely and is provided with a breast-support platform on which a patient places her breast. The breast-support platform is superimposed either on a radiosensitive film for the detection of a radiography image or an electronic detector. The image acquisition protocols include the need to compress the breast at the time of the radiography. The column has a sliding paddle capable of compressing the breast. This arrangement is hand-operated or motor-driven. The column therefore bears the following structural elements vertically, from the top downwards: means for providing a source of radiation, such as an X-ray tube; means for compression, such as the paddle means for support, such as the breast-support platform; means for detection, such as the detector.

The X-ray tube emits a first burst of X-rays through the patient's breast, and the image is acquired on, for example, an X-ray-sensitive film, positioned on the other side of the patient's breast. The operator then makes the vertical column rotate up to another position, and a second X-ray sensitive film is exposed to a second burst of X-rays. This procedure can be repeated several times to generate several images on different rolls of film. The images on the X-ray-sensitive films can then be evaluated by a physician and/or digitized and evaluated by computer. However, such a system produces a 2D image of the patient's breast. This 2D image does not give sufficient information on the presence of a tumour or a case of calcification, and often gives rise to erroneous or false positive interpretations: this is often stressful to the patient and generates excess public health costs.

To resolve this problem of erroneous or false positive interpretations, there are also now known mammography a method and apparatus that produces 3D images of the patient's breast. In this now known method and apparatus, rather than acquire an image by continuous integration of irradiation on an X-ray-sensitive film, it is preferred to sample a series of exposures by the X-ray tube along a path. The patient's breast and hence the detector, are irradiated during these successive imaging. This now known apparatus produces an image volume of the breast by tomosynthesis reconstruction. This now known apparatus has the advantage of making it less difficult to find information.

However, this now known mammography tomosynthesis method and apparatus has drawbacks. This type of apparatus has a methodology of use that is completely different from that of conventional mammography apparatus. Consequently, in order to use this now known method and apparatus, practitioners must replace existing methodologies of use by new methodologies. These new methodologies, with which practitioners have little familiarity, have not yet been adopted. This is chiefly due to the fact that these new methodologies have not been in existence for very long. Moreover, the reliability of these new methods and apparatus is not proven.

The importance of a mammography apparatus is especially great as there is a growth in studies on breast cancer detection. The frequency of use, or the rate of therapeutic examination, is a vital piece of information for such mammography apparatus. This frequency comes into play in the economic viability of the apparatus. However, the frequency of use of the now known mammography apparatus cannot be great as access to information requires more time since this information is sought sequentially in an image volume.

Another problem, which is more specific to mammography but could arise in other fields, is related to the need to be able to clinically analyze interesting micro-calcifications, sized from 100 μm to 500 μm. Consequently, the detection and characterization of the anomalies that are grounds for suspecting cancer lesions in mammography necessitates very high spatial resolution. This problem of spatial resolution is critically important in tomosynthesis mammography apparatus. These now known method and apparatus thus cannot be used to obtain images of a quality sufficient to make a fine analysis of the micro-calcifications.

### BRIEF DESCRIPTION OF THE INVENTION

An embodiment of the invention is directed at overcoming the drawbacks of the techniques described above. An embodiment of the invention is directed to a transitional apparatus that combines the two applications in a single flow of operations. This transitional apparatus is, for example, a mammography apparatus, which is well known to practitioners, while at the same time possessing the characteristics of the now known tomosynthesis apparatus. This would enable speedier adoption than in the case of an entirely different technique. This transitional apparatus enables the simultaneous acquisition and examination of a standard projection radiography image and corresponding tomosynthesis projections. In other words, this transitional apparatus carries out two examinations in one. An embodiment of the invention then provides both a 2D image formation and a 3D image formation.

In other words, an embodiment of the invention can be used to acquire simple views of projections and tomosynthesis sequences on one and the same apparatus, preferably with the same compression geometry, for an improved comparison of views.

The present invention comprises means for implementing very high spatial resolution and high contrast for cancer cells. This means optimize image quality in improving the distribution of the totality of the X-ray intensity values on different exposures during the examination. An embodiment of the present invention gives physicians greater confidence in their diagnostic tools.

An embodiment of the invention is a radiological apparatus comprising: means for emitting radiation, such as a tube fitted out with an X-ray emitting focus that emits an X-ray beam on an object, about a main direction of emission; means for detection, such as an X-ray detector situated so as to be opposite the emitter in the main direction of emission, detecting X-rays emitted during an exposure of the object; means for shifting the means for emitting radiation along a path relative to the object; the means for emitting radiation emitting intensities of radiation going through the means for a multiplicity of preliminarily determined main directions of emission along the path; and the means for detection acquiring a multiplicity of [quantities] [pieces] of X-ray image data representing the multiplicity of main directions of emission. The apparatus comprises means for the distribution of the preliminarily determined intensities of X-radiation non-uniformly on the multiplicity of main directions of emission, and means for processing the multiplicity of pieces of X-ray image data in order to obtain both a 2D image and a 3D image of the object.

The invention also relates to a method of operation of a radiological apparatus comprising: a first path of a means for emitting radiation relative to an object is determined; using the means for emitting radiation, to provide with an emitter focus, emitted radiation intensities going through the object for a multiplicity of preliminarily determined main directions of emission, along the path of the means for emitting radiation; detecting emitted radiation during an exposure of the object by means for detection situated opposite the means for emitting radiation; acquiring a multiplicity of data of radiation image data representing the multiplicity of main directions of emission; processing the multiplicity of data of radiation image data; distributing the intensities of radiation non-uniformly on the multiplicity of main directions of emission; producing a first 2D image corresponding to one of the main directions of emission, preferably the one that has received the greatest dose; and producing a first 3D image, reconstructed from the multiplicity of main directions of emission.

### BRIEF DESCRIPTION OF THE DRAWINGS

An embodiment of the invention will be understood more clearly from the following description and the accompanying figures. These figures are given by way of an indication and in no way restrict the scope of the invention, in which:

FIG. 1 is a diagrammatic view of a radiological apparatus, in particular a mammography apparatus provided with means for an embodiment of the invention;

FIG. 2 is a schematic representation of the different acquisitions of the main directions of emission, in a first path of the means for emitting radiation;

FIG. 3 is a graph showing the shape of the distribution of the intensities of the radiation in the main directions of emission;

FIG. 4 is a schematic representation of another embodiment of spreading the different acquisitions of the main directions of emission in a first path of the means for emitting radiation;

FIG. 5 is a schematic representation of the different acquisitions of the main directions of emission, in a second path of the means for emitting radiation; and

FIG. 6 is a schematic representation of the means for processing the acquisitions of the main directions of emission.

### DETAILED DESCRIPTION OF THE INVENTION

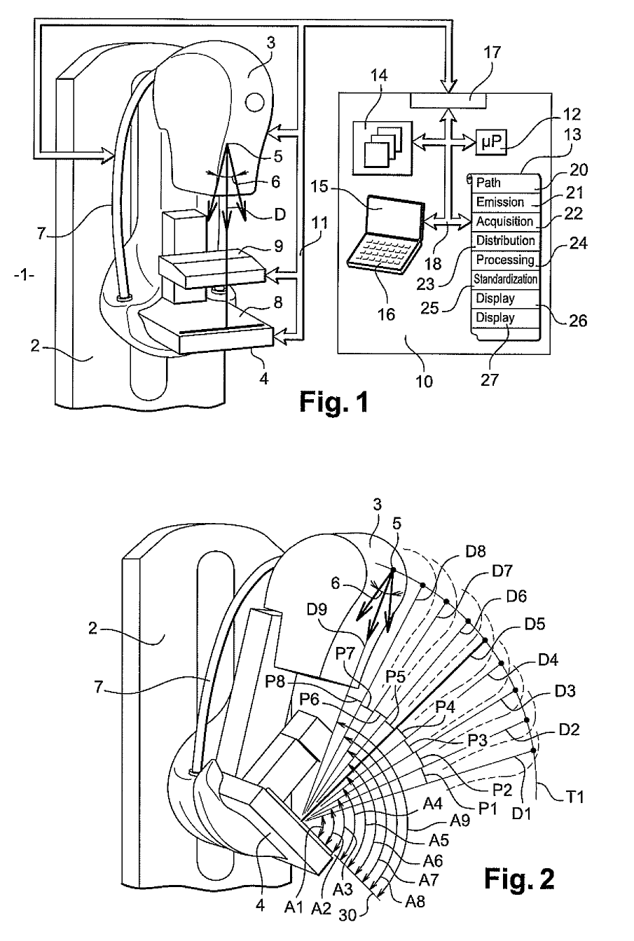

FIG. 1 shows a radiological apparatus, in particular a mammography apparatus. The mammography apparatus 1 has a vertical column 2. Vertical column 2 may be obliquely oriented. The apparatus 1 has an X-ray emitter tube 3 and a detector 4 capable of detecting the X-rays emitted by the tube 3. The tube 3 is provided with a focus 5 that is the X-ray emitting focus. This focus 5 emits an X-ray beam 6 along a main direction of emission D. The tube 3 is borne by an arm 7. An arch forms arm 7. The arm 7 is hinged on the vertical column 2 and can be used to shift the tube 3 along a path in the form of a circle arc. Other arrangements are possible, enabling the tube to move in a plane or in a sphere portion. The tube 3 can then take up different positions spread in a tilt between two extreme positions. These two positions are, for example, symmetrical to each other relative to the plane of the column 2.

The detector 4 can be an electronic detector or may be a detector with radiosensitive film for the detection of a radiography image. The detector 4 is attached to the column 2 opposite the tube 3 and in the main direction of emission D, so as to receive the X-ray beam 6.

Column 2 is provided with a breast-support table or platform 8 upon which a patient lays her breast. This breast-support platform is laid over the detector 4. The detector is placed beneath the breast-support platform 8. The detector 4 detects the X-rays that have gone through the patient's breast.

Furthermore, for reasons of stability and as well as image quality, the patient's breast needs to be compressed at the time of the radiography. Different compressive forces may be applied. These forces are applied through a compression paddle 9 which compresses the breast on the breast-support platform 8, depending on the type of examination to be made. Column 2 has a sliding paddle 9 that is capable of compressing the breast manually or is motor-driven. The paddle 9 is formed from an X-ray transparent material such as plastic. Column 2 therefore carries the following items vertically, from top to bottom: X-ray tube 3, paddle 9, breast-support platform 8 and detector 4.

While the paddle 9, the patient's breast, the platform 8 and the detector 4 are fixed, the X-ray tube 3 can take up various positions in space relative to this assembly.

After having received the beam 6 that goes through a part of the patient's body, the detector 3 emits electrical signals corresponding to the intensity of the rays received. These electrical signals may then be transmitted to a control logic unit 10 by means of an external bus 11. These electrical signals can enable control logic unit 10 to produce a 2D image and a 3D image corresponding to the part of the body analyzed. The image may be viewed by means for display, such as a screen of this control logic unit 10 or may be printed.

In order to enable each part of the patient's breast to be studied, the beam 6 may be oriented in numerous directions about the patient's breast. A user through rotation of the arm 7 may modify the position of the tube 3.

The control logic unit 10 is often made in integrated circuit form. In one example, the control logic unit 10 has a microprocessor 12, a program memory 13, a data memory 14, a display screen 15 provided with a keyboard 16 and an input/output interface 17. The microprocessor 12, the program memory 13 the data memory 14, the display screen 15 and the input/output interface 17 are interconnected by an internal bus 18.

In practice, when an action is attributed to a device, this action is performed by a microprocessor of the device controlled by instruction codes recorded in a program memory of the device. The control logic unit 10 is such a device. The program memory 13 is divided into several zones, each zone corresponding to instruction codes designed to fulfil a function of the device. According to one variant of the invention, the memory 13 comprises a zone 20 having instruction codes to set up a path of the tube 3. The memory 13 has a zone 21 comprising instruction codes to control the emission of a multiplicity of beams 6 of rays. The memory 13 has a zone 22 comprising instruction codes to acquire the data received by the detector 4. The memory 13 has a zone 23 comprising instruction codes to implement a distribution of the intensity of X-radiation on a multiplicity of beams 6 of X-rays. The memory 13 has a zone 24 comprising instruction codes to process the data received from the detector in order to obtain a 2D image and a 3D image. The memory 13 has a zone 25 comprising instruction codes to implement a standardization of the data received from the detector 4. The memory 13 has a zone 26 comprising instruction codes to view the 2D images and 3D images obtained. The memory 13 has a zone 27 comprising instruction codes to determine the mode of spreading out the multiplicity of beams.

There are several standard views classified according to ranges of values of angles. These views are given by the position of the tube 3 relative to the breast. Each of these views has a name by which it can be identified speedily and simply. For example there are MLO (mediolateral oblique) views that form part of standard exposure protocols. The list of standard views is not exhaustive.

In an operating mode, control logic unit 10 determines the path of the tube 3 as a function of standard views chosen by the practitioner. The control logic unit 10 determines the number of X-ray beams 6 to be emitted along the path of the tube 3, as can be seen in FIG. 2. The control logic unit 10 also determines a mode of spreading this number of beams 4. An example of a spread mode is shown in FIGS. 2 and 4.

Control logic unit 10 determines the X-ray intensities to be distributed on the X-ray beams 6. The X-ray intensities may be determined in line with those used in conventional mammography. In one variant, the means may be determined according to the thickness of the patient's breast.

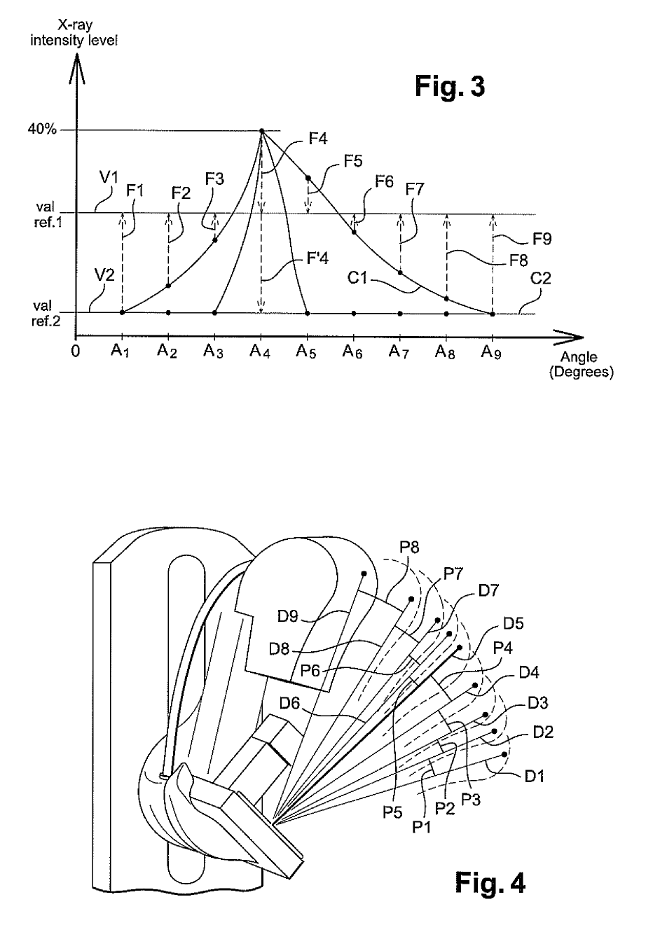

Control logic unit 10 distributes the X-ray intensities non-uniformly on the beams 4. An exemplary embodiment of such a distribution is shown in FIG. 3. To determine the mode of distribution of the X-ray intensity and the mode of spreading the beams, the control logic unit 10 preferably uses a method of pre-exposure. In this case, it activates the emission of a first beam preceding the number of beams to be emitted by the tube 3. Preferably, this first beam receives less than 5 percent of the X-ray intensity. This first beam is preferably equivalent to an automatic zero-point mode of exposure in conventional mammography.

The image produced by the control logic unit 10 corresponding to this first beam is intended for the computation of the mode of distribution of the X-ray intensities and the mode of spreading out the beams. The control logic unit 10, with a computation of less than 5 seconds, determines the mode of distribution of the X-ray intensities and the mode of spreading out the beams. This method optimizes the mode of spreading out the beams as well as the mode of distribution of the X-ray intensities by adjusting different technical parameters such as, for example, voltage, current etc.

Tube 3 emits X-ray intensities going through the patient's breast for a multiplicity of main directions of emission D, along the path. The detector 4 acquires a multiplicity of data of X-ray image data representing the multiplicity of main directions of emission D. Control logic unit 10 acquires this multiplicity of data of X-ray image data in the data memory 14. Control logic unit 10 standardizes the data of X-ray image data. Control logic unit 10 processes the data of X-ray image data in order to obtain a 2D image and a 3D image as shown in FIG. 6.

In order to obtain a full representation of the breast relative to the view chosen, the control logic unit 10 determines a second path T2 opposite the first path T1. In this second path T2, it performs the same operations as in the first path T1. The control logic unit 10 then produces two 2D and 3D images representing the complete part of the breast to be screened.

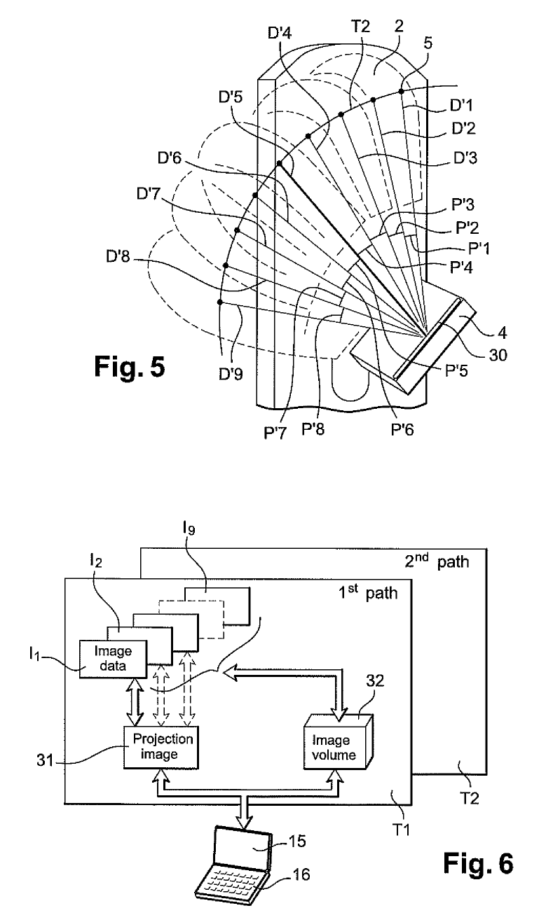

FIG. 2 shows the tube 3 emitting X-ray intensities that go through the patient's breast for a multiplicity of main directions of emission in a path. In the example of FIG. 2, the practitioner chooses to obtain images of the breast in an MLO (mediolateral oblique) view. To obtain this view, the detector 4 is placed beneath the patient's underarm and the breast is flattened vertically. In relation to this chosen standard view, the control logic unit 10 determines the two paths T1 and T2 of movement of the tube 3. These two paths are symmetrical relative to the plane of the column 2. The route taken by the focus 5 gives the shape of the two paths. In the example of FIG. 2, the first path has the shape of a circle arc. In the example of FIG. 5, the second path T2 also has the shape of a circle arc.

The control logic unit 10 proceeds by sampling a series of exposures of the tube 3. The breast and therefore the detector are thus irradiated during consecutive exposures. For these exposures, the focus of the X-ray tube occupies fixed, angularly distributed positions in space. In one example, and although this cannot be considered to be a restriction of the invention, it is provided that the angular exploration will thus be 60 degrees plus or minus 30° degrees relative to a median direction of X-radiation, which is generally vertical for a mammography.

During this exploration, a certain number of beams 6 are acquired, for example 9, 11, 13 or another number of beams, as a function of the desired precision of image reconstruction. By then applying image reconstruction algorithms of the type used in computerized tomography, it is possible to reconstruct the image in the section plane as well as other images in planes adjacent to the section plane. It is thus possible to refer to synthesis tomography where all the images are acquired in only one scan. In practice, the image in the section plane is more precise than the images in the adjacent planes when the exploration is not done on 180°. The corrections implied in the synthesis relate as much to the fact that the path of the focus of the X-ray tube is not homothetic with the position of the detector as to the fact that the detector, along the different angles of incidence, shows a tilt relative to the normal direction of projection. It is possible by computation to correct the effects of these acquisition constraints for the use of computerized tomography algorithms.

In the example of FIG. 2, the control logic unit 10 determines the number of X-ray beams 4 to be emitted by the focus 5 of the tube 4. In this example, the number of beams is nine. The multiplicity of main directions of emission is therefore represented by nine positions numbered D1 to D9. It also determines the spread of the positions of the tube to emit this multiplicity of beams 4, along the first path T1. In an example, control logic unit 10 evenly spreads the positions of emission of the tube 3 on the first path T1.

Control logic unit 10 determines the mode of non-uniform distribution of the totality of the X-radiation intensities, commonly called a dose, between the different main directions of emission of the two paths T1 and T2. This non-uniform distribution gives high contrast for cancer cells. The dose is preferably equal to the dose used in the prior art to obtain the two radiography projections in standard mammography. In the prior art, the two radiography projections each receive 50% of the dose. The two radiography projections represent two standard views.

In the example of FIG. 2, this dose is distributed as a function of the angle A1 to A8 respectively formed by each of the main directions of emission D1 to D9 with a normal 30 of the detector 4, as can be seen in FIG. 3. The dose is distributed on both paths T1 and T2. The example of FIG. 2 shows a mode of distribution of the dose on the path T1. The example of FIG. 5 shows a mode of distribution of the dose on the path T2. In an embodiment, the control logic unit 10 assigns a strong dose to the main direction of emission, preferably representing a standard view, in each path.

In one variant, control logic unit 10 may assign a heavier dose to the main direction of emission that is substantially perpendicular to the plane of the detector 4. Control logic 10 may also assign a heavier dose to a main direction of emission as a function of the practitioner's prerogatives and technical constraints of the apparatus. Consequently, any of the main directions of emission may receive the heaviest dose.

In one example, for each path T1 and T2, the control logic unit 10 determines the preferred main direction of emission. In one example, control logic 10 assigns 40 percent of the dose to each of the two main preferred directions of the two paths T1 and T2. Control logic 10 distributes the rest of the dose, which is 20 percent of the dose, preferably non-uniformly in the other remaining main directions of emission of the two paths T1 and T2.

In the example of FIG. 2, the main direction of emission is the one that separates the number of main directions of emission into two equal parts. Control logic unit 10 assigns a greater dose to the main direction of emission. The main direction of emission is represented by D5. As compared with the other directions that are represented by thin lines, D5 is represented by a bold line in order to show that it receives a greater dose than the other main directions of emission.

Control logic unit 10 may first of all control the emission of the main direction of emission D5 before emitting in the other directions. Control logic unit 10 also controls the emission of the directions from D1 to D4, then D6 to D7, before emitting in the direction D5. Control logic unit 10 also controls only emission in the direction of one of the equal parts, such as D1 to D4 or D6 to D9, plus the direction D5. In this case, it determines the directions of the other equal part in considering D5 as the bisectrix of the two parts.

Two consecutive directions form an angular step. In the example of FIG. 2, the angular step is uniform. The angular step P1 is formed by the directions D1 and D2 and so on and so forth until the angular step P8 which is formed by the directions D8 and D9. The angular steps P1 to P8 form a 30-degree angle in the example of FIG. 2. The uniformity of the steps is given by the spreading mode determined by the control logic unit 10. When the spreading mode is regular, the angular steps are uniform. And when the spreading mode is irregular, the angular steps are non-uniform.

FIG. 3 shows the dose distribution mode in an embodiment. In this example, the y-axis is formed by the level of x-ray intensities or dose level and the x-axis is formed by the angles A1 to A8 respectively formed by each of the main directions of emission D1 to D9 with the normal 30 to the detector 4, as can be seen in FIG. 2. The dose is distributed non-uniformly on the totality of the main directions of emission D1 to D9. Here, the main direction of emission D4 is the one receiving the greatest dose. In one example, the direction D4 receives 40% of the total dose. In this example, the control logic unit 10 commands the emission first of all in the direction D4. Then, control logic unit 10 commands the emission in the other directions in assigning them a low dose, when the angle of emission is distant from the angle A4 formed with the direction D4. It augments the dose when the angle of direction approaches the angle A4 formed with the direction D4. Thus, the curve C1 of the distribution of the dose between the directions D1 to D9 has a hyperbolic shape.

The control logic unit 10 also achieves an example of standardization as can be seen in FIG. 3. The dose level of each of the directions D1 to D9 is reduced to a standardization value that herein is the reference value V1. The reference value V1 for the standardization is determined as a function of an optimal image quality. A filtering circuit can be used to implement this standardization. Another type of standardisation circuit can be used to implement a noise-free standardization. This standardisation is shown in FIG. 3 by the arrows F1 to F9 formed by dashes. Arrows F1 to F9 respectively shows the sense of standardization of the X-radiation intensities in the main directions D1 to D9.

In one variant, the control logic unit 10 may assign 40 percent of the dose to the direction D4 and assign a uniform dose to the other directions as can be seen in the shape of the dose-distribution curve C2. In this case, the control logic unit 10 may choose a standardization value that is the reference value V2 representing the uniform intensity level of the main directions of emission D1 to D3 and D5 to D9.

FIG. 4 shows another mode of non-uniform spreading of the main directions of emission D1 to D9 on the first path T1. The angular steps P1 to P9, formed respectively by two consecutive main directions of emission D1 to D9, are irregular. In the example of FIG. 4, the angular steps P8 and P4 form an angle of 60 degrees. The angular steps P7 and P3 form an angle of 30 degrees and the other angular steps form an angle of 15 degrees. The spread of the directions D1 to D9 can be according to other types of angular steps that may be irregular or regular. The angular steps may have other angles that are different from those of the disclosed examples.

FIG. 5 shows the spread and the distribution of the dose in the different directions D′1 to D′9, along the second path T2. The directions D′1 to D′9 are respectively spread in an angular step P′1 to P′9. This angular step is constant. The spread mode then chosen by the control logic unit 10 is regular.

The control logic unit determines the mode of distribution of the dose in each of the directions D′1 to D′9. The control logic unit determines which of the directions is given preference by the practitioner. Based on this determining, it grants this direction 40 percent of the dose. In the example of FIG. 5, this direction is the direction D′5. Since each of the directions D5 and D′5, of the two respective paths T1 and T2, receives 40 percent of the dose, the control logic unit 10 carries out a uniform or non-uniform distribution of the remaining 20 percent of the dose on the remaining directions of the two paths T1 and T2.

The directions D′1 to D′4 and D′6 to D′9 of the second path T2 and the directions D1 to D4 and D6 to D9 of the first path T1 have their dose that, in one example, depends on the respective angles that they form with the normal 30 of the detector 4.

The detector 4 firstly acquires all the X-ray image data representing the main directions of emission D1 to D9 of the first path T1. Secondly, the detector 4 acquires the X-ray image data representing the main directions of emission D′1 to D′9 of the second path T2. The processing of this data of image data is shown in FIG. 6.

FIG. 6 gives a schematic view of an example of image processing used to obtain a 2D image and a 3D image. The mode of processing image data for the two paths T1 and T2 is identical. Hence, only the mode of processing data image given by the first path T1 will be considered here. Each of the pieces of X-ray image data T1 to 19 given by the detector respectively represents the main directions of emission D1 to D9. Depending on the desired main direction, the control logic unit 10 sends the corresponding image data to the first processing unit 31. This first processing unit 31 produces a 2D image. This 2D image is the projection radiography image produced by presently used mammography apparatus. This projection image is shown on the display screen 16.

All the data of image data T1 to 19 are used during a tomosynthesis reconstruction. The image of image data is sent by the control logic unit 10 to a processing unit 32. The processing unit 32 gives a digital volume. Processing unit 32, using a tomosynthesis technique, and on the basis of a small number of 2D projections or data of image data, spread over a restricted angular domain and acquired on a digital detector, is used to reconstruct the 3D volume of the breast under examination.

The method may comprise one or more of the following characteristics: determining a second path of the tube, relative to the object and opposite the first path; emitting preliminarily determined intensities of X-radiation, going through the object for a multiplicity of preliminarily determined main directions of emission, along the path of the tub; distributing intensities of X-radiation non-uniformly on the multiplicity of main directions of emission; producing a second 2D image, corresponding to one of the main directions of emission; producing a second 3D image corresponding to the multiplicity of main directions of emission; representing the 3D image by a tomosynthesis reconstruction; giving preference to a main direction of emission relative to the multiplicity of main directions of emission, for each path; distributing 40% of the intensities of x-radiation on the preferred main direction of emission, for each path; and distributing the remaining 20% of the intensities of X-radiation on the remaining main directions of emission of the two paths; detecting the intensity of all X-radiation of the preferred main directions of emission before detecting the intensities of X-radiation of the remaining main directions of emission, for each path.

The method may comprise one or more of the following characteristics: detecting the intensity of X-radiation of the preferred main direction of emission after having detected the intensities of X-radiation of the remaining main directions of emission, for each path; causing the multiplicity of main directions of emission to be preceded by an unspecified main direction of emission with a low intensity of X-radiation; determining, by computation, the mode of distribution of the intensities of X-radiation and the mode of spreading out the multiplicity of main directions of emission of the two paths, from the unspecified main direction of emission.

The method and apparatus enables a practitioner to obtain access, at a glance, to a clear image that has high contrast at every point, without needing to adjust the display. With such an image, the radiologist should be capable of identifying all the clinical signs in perceiving the relationships between the different components of the image. If this image is not clear, the radiologist can access a 3D image through a key of the keyboard 16.

In addition, while an embodiment of the invention has been described with reference to exemplary embodiments, it will be understood by those skilled in the art that various changes may be made in the function and/or way and/or result and equivalents may be substituted for elements thereof without departing from the scope and extent of the invention. In addition, many modifications may be made to adapt a particular situation or material to the teachings of the invention without departing from the essential scope thereof. Therefore, it is intended that the invention not be limited to a particular embodiment disclosed for carrying out this invention, but that the invention will include all embodiments falling within the scope of the appended claims. Moreover, the use of the terms first, second, etc. or steps do not denote any order or importance, but rather the terms first, second, etc. or steps are used to distinguish one element or feature from another. Furthermore, the use of the terms a, an, etc. do not denote a limitation of quantity, but rather denote the presence of at least one of the referenced element or feature.

What is claimed is:

1. A radiological apparatus comprising:
means for emitting radiation having an emitting focus that emits a beam of radiation on an object, about a main direction of emission;
means for detection situated opposite the means for emission in the main direction of emission, detecting the beam emitted during an exposure of the object;
means for shifting the means for emitting radiation along a path relative to the object,
the means for emitting providing emitting intensities of radiation going through the means for emitting for a multiplicity of preliminarily determined main directions of emission along the path;
the means for detection acquiring a multiplicity of data of image data representing the multiplicity of main directions of emission;
means for the distribution of the preliminarily determined intensities of radiation non-uniformly on the multiplicity of main directions of emission; and
means for processing the multiplicity of data of image data in order to obtain both a 2D image and a 3D image of the object.

2. The apparatus according to claim 1 wherein the means for processing the multiplicity of data of image data comprises means for a tomosynthesis reconstruction to obtain a 3D image.

3. The apparatus according to claim 1 wherein the means of distribution of the radiation intensities are designed to adjust the intensities as a function of the angle formed by each of the main directions of emission with a normal to the means for detection.

4. The apparatus according to claim 2 wherein the means of distribution of the radiation intensities are designed to adjust the intensities as a function of the angle formed by each of the main directions of emission with a normal to the means for detection.

5. The apparatus according to claim 1 wherein the means of distribution of the radiation intensities are designed to make the means for emission to emit in main directions of emission that are spread according to a non-uniform step value along the path.

6. The apparatus according to claim 2 wherein the means of distribution of the radiation intensities are designed to make the means for emission to emit in main directions of emission that are spread according to a non-uniform step value along the path.

7. The apparatus according to claim 3 wherein the means of distribution of the radiation intensities are designed to make the means for emission to emit in main directions of emission that are spread according to a non-uniform step value along the path.

8. The apparatus according to claim 1 wherein the means of distribution assign a greater intensity of radiation to the main direction of emission that is substantially perpendicular to the plane of the means for detection.

9. The apparatus according to claim 2 wherein the means of distribution assign a greater intensity of radiation to the main direction of emission that is substantially perpendicular to the plane of the means for detection.

10. The apparatus according to claim 3 wherein the means of distribution assign a greater intensity of radiation to the main direction of emission that is substantially perpendicular to the plane of the means for detection.

11. The apparatus according to claim 4 wherein the means of distribution assign a greater intensity of radiation to the main direction of emission that is substantially perpendicular to the plane of the means for detection.

12. The apparatus according to claim 1 wherein the means of distribution assign a greater intensity of radiation to the main direction of emission which divides the multiplicity of main directions of emission into two parts, equal in number of directions.

13. The apparatus according to claim 2 wherein the means of distribution assign a greater intensity of radiation to the main direction of emission which divides the multiplicity of main directions of emission into two parts, equal in number of directions.

14. The apparatus according to claim 3 wherein the means of distribution assign a greater intensity of radiation to the main direction of emission which divides the multiplicity of main directions of emission into two parts, equal in number of directions.

15. The apparatus according to claim 4 wherein the means of distribution assign a greater intensity of radiation to the main direction of emission which divides the multiplicity of main directions of emission into two parts, equal in number of directions.

16. The apparatus according to claim 5 wherein the means of distribution assign a greater intensity of radiation to the main direction of emission which divides the multiplicity of main directions of emission into two parts, equal in number of directions.

17. The apparatus according to claim 1 comprising means for standardization of the radiation intensities.

18. The apparatus according to claim 2 comprising means for standardization of the radiation intensities.

19. The apparatus according to claim 3 comprising means for standardization of the radiation intensities.

20. The apparatus according to claim 4 comprising means for standardization of the radiation intensities.

21. The apparatus according to claim 5 comprising means for standardization of the radiation intensities.

22. The apparatus according to claim 6 comprising means for standardization of the radiation intensities.

23. A method of operation of a radiological apparatus comprising:
determining a first path of a means for emission of radiation relative to an object;
emitting radiation intensities through the object for a multiplicity of preliminarily determined main directions of emission, along the path of the means for emission;
detecting the radiation emitted during an exposure of the object by means for detection situated opposite the means for emission;
acquiring a multiplicity of data of image data representing the multiplicity of main directions of emission;
processing the multiplicity of pieces of image data;
distributing the intensities of radiation non-uniformly on the multiplicity of main directions of emission;
producing a first 2D image corresponding to one of the main directions of emission; and
producing a first 3D image, reconstructed from the multiplicity of main directions of emission.

24. The method according to claim 23 comprising producing the first 2D image from the image that has received the greatest dose of radiation.

25. The method according to claim 23 comprising:
determining a second path of the means for emission, relative to the object and opposite the first path;
emitting preliminarily determined intensities of radiation, through the object for a multiplicity of preliminarily determined main directions of emission, along the path of the means for emission;
distributing intensities of X-radiation non-uniformly on the multiplicity of main directions of emission;
producing a second 2D image, corresponding to one of the main directions of emission; and
producing a second 3D image corresponding to the multiplicity of main directions of emission.

26. The method according to claim 24 comprising:
determining a second path of the means for emission, relative to the object and opposite the first path;
emitting preliminarily determined intensities of radiation, through the object for a multiplicity of preliminarily determined main directions of emission, along the path of the means for emission;
distributing intensities of X-radiation non-uniformly on the multiplicity of main directions of emission;
producing a second 2D image, corresponding to one of the main directions of emission; and
producing a second 3D image corresponding to the multiplicity of main directions of emission.

27. The method according to claim 23 comprising achieving the representation of the 3D image by a tomosynthesis reconstruction.

28. The method according to claim 24 comprising achieving the representation of the 3D image by a tomosynthesis reconstruction.

29. The method according to claim 25 comprising achieving the representation of the 3D image by a tomosynthesis reconstruction.

30. The method according to claim 23 comprising:
giving preference to a main direction of emission relative to the multiplicity of main directions of emission, for each path;
distributing 40% of the intensities of radiation on the preferred main direction of emission, for each path; and
distributing the remaining 20% of the intensities of radiation on the remaining main directions of emission of the two paths.

31. The method according to claim 24 comprising:
giving preference to a main direction of emission relative to the multiplicity of main directions of emission, for each path;
distributing 40% of the intensities of radiation on the preferred main direction of emission, for each path; and
distributing the remaining 20% of the intensities of radiation on the remaining main directions of emission of the two paths.

32. The method according to claim 25 comprising:
giving preference to a main direction of emission relative to the multiplicity of main directions of emission, for each path;
distributing 40% of the intensities of radiation on the preferred main direction of emission, for each path; and
distributing the remaining 20% of the intensities of radiation on the remaining main directions of emission of the two paths.

33. The method according to claim 26 comprising:
giving preference to a main direction of emission relative to the multiplicity of main directions of emission, for each path;
distributing 40% of the intensities of radiation on the preferred main direction of emission, for each path; and
distributing the remaining 20% of the intensities of radiation on the remaining main directions of emission of the two paths.

34. The method according to claim 27 comprising:
giving preference to a main direction of emission relative to the multiplicity of main directions of emission, for each path;
distributing 40% of the intensities of radiation on the preferred main direction of emission, for each path; and
distributing the remaining 20% of the intensities of radiation on the remaining main directions of emission of the two paths.

35. The method according to claim 23 comprising detecting first the intensity of radiation of the preferred main directions of emission, before detection of the intensities of radiation of the remaining main directions of emission, for each path.

36. The method according to claim 24 comprising detecting first the intensity of radiation of the preferred main directions of emission, before detection of the intensities of radiation of the remaining main directions of emission, for each path.

37. The method according to claim 25 comprising detecting first the intensity of radiation of the preferred main directions of emission, before detection of the intensities of radiation of the remaining main directions of emission, for each path.

38. The method according to claim 26 comprising detecting first the intensity of radiation of the preferred main directions of emission, before detection of the intensities of radiation of the remaining main directions of emission, for each path.

39. The method according to claim 27 comprising detecting first the intensity of radiation of the preferred main directions of emission, before detection of the intensities of radiation of the remaining main directions of emission, for each path.

40. The method according to claim 28 comprising detecting first the intensity of radiation of the preferred main directions of emission, before detection of the intensities of radiation of the remaining main directions of emission, for each path.

41. The method according to claim 29 comprising detecting first the intensity of radiation of the preferred main directions of emission, before detection of the intensities of radiation of the remaining main directions of emission, for each path.

42. The method according to claim 30 comprising detecting first the intensity of radiation of the preferred main directions of emission, before detection of the intensities of radiation of the remaining main directions of emission, for each path.

43. The method according to claim 23 comprising detecting the intensity of radiation of the preferred main direction of emission after detection of the intensities of radiation of the remaining main directions of emission, for each path.

44. The method according to claim 24 comprising detecting the intensity of radiation of the preferred main direction of emission after detection of the intensities of radiation of the remaining main directions of emission, for each path.

45. The method according to claim 25 comprising detecting the intensity of radiation of the preferred main direction of emission after detection of the intensities of radiation of the remaining main directions of emission, for each path.

46. The method according to claim 26 comprising detecting the intensity of radiation of the preferred main direction of emission after detection of the intensities of radiation of the remaining main directions of emission, for each path.

47. The method according to claim 27 comprising detecting the intensity of radiation of the preferred main direction of emission after detection of the intensities of radiation of the remaining main directions of emission, for each path.

48. The method according to claim 28 comprising detecting the intensity of radiation of the preferred main direction of emission after detection of the intensities of radiation of the remaining main directions of emission, for each path.

49. The method according to claim 29 comprising detecting the intensity of radiation of the preferred main direction of emission after detection of the intensities of radiation of the remaining main directions of emission, for each path.

50. The method according to claim 30 comprising detecting the intensity of radiation of the preferred main direction of emission after detection of the intensities of radiation of the remaining main directions of emission, for each path.

51. The method according to claim 31 comprising detecting the intensity of radiation of the preferred main direction of emission after detection of the intensities of radiation of the remaining main directions of emission, for each path.

52. The method according to claim 32 comprising detecting the intensity of radiation of the preferred main direction of emission after detection of the intensities of radiation of the remaining main directions of emission, for each path.

53. The method according to claim 33 comprising detecting the intensity of radiation of the preferred main direction of emission after detection of the intensities of radiation of the remaining main directions of emission, for each path.

54. The method according to claim 34 comprising detecting the intensity of radiation of the preferred main direction of emission after detection of the intensities of radiation of the remaining main directions of emission, for each path.

55. The method according to claim 35 comprising detecting the intensity of radiation of the preferred main direction of emission after detection of the intensities of radiation of the remaining main directions of emission, for each path.

56. The method according to claim 23 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

57. The method according to claim 24 comprising: preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

58. The method according to claim 25 comprising: preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

59. The method according to claim 26 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

60. The method according to claim 27 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

61. The method according to claim 28 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

62. The method according to claim 29 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

63. The method according to claim 30 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

64. The method according to claim 31 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

65. The method according to claim 32 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

66. The method according to claim 33 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

67. The method according to claim 34 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

68. The method according to claim 35 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

69. The method according to claim 36 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

70. The method according to claim 37 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

71. The method according to claim 38 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

72. The method according to claim 39 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

73. The method according to claim 40 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

74. The method according to claim 41 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

75. The method according to claim 42 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

76. The method according to claim 43 comprising:
preceding the multiplicity of main directions of emission by an unspecified main direction of emission with a low intensity of radiation; and
determining by computation the mode of distribution of the intensities of radiation and the mode of spreading the multiplicity of main directions of emission of the two paths from the unspecified main direction of emission.

77. A computer program comprising program code means for implementing the method according to claim 1 when the program runs on a computer.

78. A computer program product comprising a computer useable medium having computer readable program code means embodied in the medium, the computer readable program code means implementing the method according to claim 1.

79. An article of manufacture for use with a computer system, the article of manufacture comprising a computer readable medium having computer readable program code means embodied in the medium, the program code means implementing the method according to claim 1.

80. A program storage device readable by a machine tangibly embodying a program of instructions executable by the machine to perform the method according to claim 1.
